(19) United States
(12) Patent Application Publication
(10) Pub. No.: US 2025/0326709 A1
(43) Pub. Date: Oct. 23, 2025

(54) THE MICROWAVE-ASSISTED CATALYTIC AMIDATION OF ORGANIC AMINES WITH CARBOXYLIC ACIDS

(71) Applicant: Purdue Research Foundation, West Lafayette, IN (US)

(72) Inventors: David Harley Thompson, West Lafayette, IN (US); Giulia Murbach de Oliveira, West Lafayette, IN (US); Shruti Biyani, Woodbridge, NJ (US)

(73) Assignee: Purdue Research Foundation, West Lafayette, IN (US)

(21) Appl. No.: 19/184,279

(22) Filed: Apr. 21, 2025

Related U.S. Application Data
(60) Provisional application No. 63/637,005, filed on Apr. 22, 2024.

(51) Int. Cl. C07C 231/02; B01J 19/12; B01J 20/18; B01J 21/02; B01J 27/10

(52) U.S. Cl. CPC C07C 231/02; B01J 19/126; B01J 20/183; B01J 21/02; B01J 27/10

(57) ABSTRACT

A microwave-assisted catalytic amidation of amines with acids using a combination of a catalyst and an additive in the presence of a bio-renewable green organic solvent.

### CROSS-REFERENCE TO RELATED APPLICATIONS

This application claims priority to U.S. provisional patent application No. 63/637,005 which was filed Apr. 22, 2024, and which is hereby incorporated by reference in its entirety.

### GOVERNMENT RIGHTS

This invention was made with government support under 2132142 awarded by the National Science Foundation. The government has certain rights in the invention.

### TECHNICAL FIELD

The present disclosure relates to a microwave-assisted catalytic amidation of amines with acids using a combination of a catalyst and an additive in the presence of a bio-renewable green organic solvent. Compared to conventional heating, the microwave process requires less energy inputs and reduces the reaction time drastically.

### BACKGROUND

This section introduces aspects that may help facilitate a better understanding of the disclosure. Accordingly, these statements are to be read in this light and are not to be construed as admissions about what is or is not prior art.

The formation of amides is vital in the pharmaceutical industry, with amides being present in 25% of all drug molecules and the amide bond formation being considered one of the most used transformations in medicinal chemistry. The most utilized methods to produce amides were studied, finding that stoichiometric amidation with coupling reagents and acid chloride-mediated transformations represented 65% of the amidations reported in the Reaxys database between 2017-2018 (Nature Catalysis, 2019, 2, 10-17). Advances have been made in the catalytic formation of amides utilizing zirconium, copper (Chem Commun, 2012, 48, 666-668; Chem-Eur J, 2012, 18, 3822-3826) and boronic acids/boronates (Chem Soc Rev, 2019, 48, 3475-3496), as well as biocatalysts (Green Chem. 2023, 25, 2958-2970). Boronic acids are air-stable and exhibit low toxicity (Future Medicinal Chemistry, 2009, 1, 1275-1288).

The studies in Org Biomol Chem, 2013, 11, 1822-1839, reported that an acid precursor, 4-(chloromethyl)benzoic acid, yielded only 20% amide coupled product in stoichiometric reactions with 3-bromo-4-methylaniline unless the acid was converted to the acyl chloride to improve yield. The use of benzoic acid and aniline as starting materials underscores the difficulty of forming amides when both starting materials are aromatic, primarily due to the aniline nitrogen's lone pair with the adjacent π system.

It was an object of the present invention to provide a method for amide formation by reacting an amine with an acid using a suitable catalyst and a microwave radiation that requires less energy input and reaction time and provides excellent yield.

### SUMMARY

Provided is a method of a catalytic amidation, which method comprises reacting an amine with an acid using a catalyst selected from a boronic acid, a borate, a boric acid, a boronic acid ester, and a Lewis acid, optionally in combination with an amine N-oxide additive, using a microwave radiation. The amine can be an aryl amine or an alkyl amine. The acid can be an aryl acid or an alkyl acid. The method further comprises the use of an organic solvent. The organic solvent can be 2-methyl tetrahydrofuran.

The catalyst can be selected from triphenyl borate, 2,4-bis(trifluoromethyl) phenyl boronic acid, phenylboronic acid pinacol ester, bis(catecholato)diboron, tetrahydroxy diboron, butylboronic acid, boric anhydride, trimethyl borate, and boric acid. The catalyst can be a Lewis acid catalyst. The catalyst can be bis(cyclopentadienyl)zirconium(IV) dichloride. In some embodiments, the catalyst is 2,4-bis(trifluoromethyl)phenylboronic acid.

The method can be carried out with or without using an additive. Any suitable amine N-oxide can be used as an additive. The additive can selected from trimethylamine N-oxide, isoquinoline N-oxide, pyridine N-oxide, 2-aminopyridine N-oxide, 2-(4-methoxyphenyl)pyridine N-oxide, 2-mercaptopyridine N-oxide, 2-methyl-4-nitropyridine N-oxide, 2-pyridinol 1-oxide, 2,6-dichloropyridine N-oxide, 4-chloropyridine N-oxide, 4-cyanopyridine N-oxide, 4-methylpyridine N-oxide, 4-phenylpyridine N-oxide, 4-(dimethylamino)pyridine N-oxide, 4-methylmorpholine N-oxide, N-tert-butyl-alpha-(4-pyridyl-1-oxide)nitrone, N-tert-butyl-alpha-4-phenylnitrone, and Resazurin sodium salt. In some embodiments, the additive is trimethylamine N-oxide.

The method further comprises a step of removing or complexing water. The water can be removed by using a drying agent selected from a molecular sieve and CaO or other well-known methods. In some embodiments, the drying agent is a molecular sieve. The amount of the catalyst can be loaded in a range from about 0.5 mol % to about 50 mol %. The amount of the catalyst and additive used can be in a ratio of about 1:1 (such as 1:1). The aryl amine and aryl acid can be used in a ratio of about 1:1. The method can be carried out at in a temperature range from about 20° C. to about 180° C. The method can be carried out in a time period of about 1 minute to about 150 minutes.

### BRIEF DESCRIPTION OF DRAWINGS

FIG. 1 shows a table for a heat map for the first round of design of experiments and high throughput experimentation (DoE-THE) depicting signal-to-noise ratios (S/N).

FIG. 2 shows a table for a heat map for the second round of DoE-THE depicting signal-to-noise ratios (S/N).

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The formation of amide is one of the most essential transformations in the pharmaceutical industry. Most synthetic procedures include the use of stoichiometric poor atom economy reagents. Due to their inherently low reactivity, these limitations are often compounded when condensing aryl amines with aryl acids.

In view of the above, provided is a catalytic amidation that can transform amines and carboxylic acids to amides, including sterically hindered and unreactive aromatic and secondary amines. The method can involve the use of (i) a suitable catalyst that can improve atom economy, (ii) a biorenewable solvent, (iii) a high substrate loading, and (iv) an energy-efficient microwave heating.

Provided is a method of a catalytic amidation, which method comprises reacting an amine with an acid using a catalyst selected from a boronic acid, a borate, a boric acid, a boronic acid ester, and a Lewis acid, optionally in combination with amine N-oxide additive, using a microwave radiation.

The method can be carried out using a microwave synthesizer. Microwave synthesizers can allow for a much faster and homogeneous heating of the solvent mixture compared to the conventional heating methods, because of the “inside out” phenomenon that has been associated with reduced reaction times. Microwave heating can be more efficient and safer when dealing with temperatures above the boiling point of a solvent since the vapors are contained within the closed system, and the applied power is regulated by a pressure sensor.

Any suitable amine can be used. The amine can be an aryl amine or an alkyl amine. Any suitable acid can be used. The acid can be an aryl carboxylic acid or an alkyl carboxylic acid. The catalyst can be selected from a boronic acid, a borate, a boric acid, a boronic acid ester, and a Lewis acid. In some embodiments, the catalyst can be a Lewis acid catalyst. In some embodiments, the catalyst can be triphenyl borate, 2,4-bis(trifluoromethyl) phenyl boronic acid, phenylboronic acid pinacol ester, bis(catecholato)diboron, tetrahydroxy diboron, butylboronic acid, boric anhydride, trimethyl borate, or boric acid. In some embodiments, the catalyst is 2,4-bis(trifluoromethyl)phenylboronic acid. In some embodiments, the catalyst is bis(cyclopentadienyl)zirconium(IV) dichloride. The catalyst can be used in a concentration of about 0.5 mol % to about 50 mol %, such as about 0.5 mol % to 50 mol %, 0.5 mol % to about 50 mol %, or 0.5 mol % to 50 mol %. In some embodiments, the catalyst amount used is about 15 mol % (such as 15 mol %). In some embodiments, the catalyst amount used is about 25 mol % (such as 25 mol %).

The method can be carried out with or without using an additive. The additive can be used optionally with the catalyst listed above. Any suitable N-oxide can be used as an additive. In some embodiments, N-oxide can be amine N-oxide. The additive can selected from trimethylamine N-oxide, isoquinoline N-oxide, pyridine N-oxide, 2-aminopyridine N-oxide, 2-(4-methoxyphenyl)pyridine N-oxide, 2-mercaptopyridine N-oxide, 2-methyl-4-nitropyridine N-oxide, 2-pyridinol 1-oxide, 2,6-dichloropyridine N-oxide, 4-chloropyridine N-oxide, 4-cyanopyridine N-oxide, 4-methylpyridine N-oxide, 4-phenylpyridine N-oxide, 4-(dimethylamino)pyridine N-oxide, 4-methylmorpholine N-oxide, N-tert-butyl-alpha-(4-pyridyl-1-oxide)nitrone, N-tert-butyl-alpha-4-phenylnitrone, and Resazurin sodium salt. In some embodiments, the additive is trimethylamine N-oxide (TMAO). The additive can be used in a concentration of about 0.5 mol % to about 50 mol %, such as about 0.5 mol % to 50 mol %, 0.5 mol % to about 50 mol %, or 0.5 mol % to 50 mol %. In some embodiments, the amount of additive used is about 15 mol % (such as 15 mol %). In some embodiments, the amount of additive used is about 25 mol % (such as 25 mol %). In some embodiments, the amount of the catalyst and additive used can be in a ratio of about 1:1 (such as 1:1).

In some embodiments, the aryl amine and aryl acid can be used in a ratio of about 1:1 to about 1:1.1, such as about 1:1 to 1:1.1, 1:1 to about 1:1.1 or 1:1 to 1:1.1. In some embodiments, the aryl amine and aryl acid is used in a ratio of about 1:1 (such as 1:1).

The method further comprises the use of an organic solvent. Any suitable biorenewable organic solvent can be used. The solvent can have a low water content. In some embodiments, the organic solvent can be 2-methyl tetrahydrofuran (2-MeTHF) or toluene. In some embodiments, the organic solvent is 2-methyl tetrahydrofuran (2-MeTHF). The substrate concentrations in the solvent can range from about 0.05 M to about 2 M, such as about 0.05 M to 2 M, 0.05 M to about 2 M, or 0.05 M to 2 M. In some embodiments, the substrate concentrations in the solvent can be about 0.25 M (such as 0.25M). In some embodiments, the substrate concentrations in the solvent can be of about 0.5 M (such as 0.5M).

The method can further comprise a step of removing water or complexing water. The water can be removed by using a drying agent or other well-known methods. The drying agent can be a molecular sieve or calcium oxide (CaO). In some embodiments, the drying agent is a molecular sieve. The molecular sieve can be 3 Å or 4 Å. The amount of the drying agent used can be about 0.5 g/mL solvent to about 2 g/mL solvent, such as about 0.5 g/mL solvent to 2 g/mL solvent, 0.5 g/mL solvent to about 2 g/mL solvent or 0.5 g/mL solvent to 2 g/mL solvent.

The reaction under microwave radiation can be carried out at in a temperature range from about 20° C. to about 180° C., such as 20° C. to about 180° C., about 20° C. to 180° C., or 20° C. to 180° C. The method can be carried out in a time period of about 1 minute to about 150 minutes, such as about 1 minute to 150 minutes, 1 minute to about 150 minutes, or 1 minute to 150 minutes. In some embodiments, the time period is about 10 minutes. In some embodiments, the time period is about 15 minutes. In some embodiments, the time period is about 30 minutes. In some embodiments, the time period is about 1 hour. In some embodiments, the time period is about 2 hours.

The method can convert sterically hindered amines and carboxylic acids to the corresponding N-arylbenzamides, including previously reported unreactive aromatic and secondary amines, such as compounds 24, 25, 26, and 27 in good to excellent yields of 70%, 95%, 84%, and 86%, respectively whereas the reported yields are 0%, 0%, 0%, and 38%, respectively.

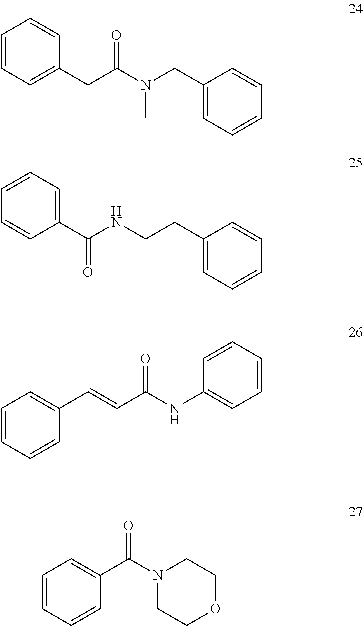 [SMILES: CN(Cc1ccccc1)C(=O)Cc1ccccc1 ; O=C(NCCc1ccccc1)c1ccccc1 ; O=C(/C=C/c1ccccc1)Nc1ccccc1 ; O=C(c1ccccc1)N1CCOCC1]

The term “alkyl” refers to substituted and unsubstituted straight-chain and branched alkyl groups and cycloalkyl groups having from 1 to about 20 carbon atoms (e.g., C₁-C₂₀), 1 to 12 carbons (e.g., C₁-C₁₂), 1 to 8 carbon atoms (e.g., C₁-C₈), or, in some embodiments, from 1 to 6 carbon atoms (e.g., C₁-C₆). Examples of straight-chain alkyl groups include those with from 1 to 8 carbon atoms, such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “optionally substituted” or “optional substituents” means that the groups in question are either unsubstituted or substituted with one or more of the substituents specified. When the groups in question are substituted with more than one substituent, the substituents may be the same or different. The terms “independently,” “independently are,” and “independently selected from” mean that the groups in question may be the same or different. Certain may occur more than once in the structure, and upon such occurrence, each term shall be defined independently of the other.

The term “aryl” refers to substituted and unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (e.g., C₆-C₁₄) or from 6 to 10 carbon atoms (e.g., C₆-C₁₀) in the ring portions of the groups. Aryl groups can be unsubstituted or substituted, as defined herein. Representative substituted aryl groups can be mono-substituted or substituted more than once, such as, but not limited to, a phenyl ring substituted with 2-, 3-, 4-, 5-, or 6-substituents or 2-8 substituted naphthyl groups, which can be substituted with carbon or non-carbon groups such as those listed herein.

The term “amine” refers to primary, secondary, and tertiary amines having, e.g., the formula N(group)₃ wherein each group can independently be H or non-H, such as alkyl, aryl, and the like. Amines include, but are not limited to, R—NH₂, for example, alkylamines, arylamines, alkylarylamines; R₂NH, wherein each R is independently selected, such as dialkylamines, diarylamines, aralkylamines, heterocyclylamines and the like; and R₃N, wherein each R is independently selected, such as trialkylamines, dialkylarylamines, alkyldiarylamines, triarylamines, and the like. The term “amine” also includes ammonium ions as used herein. The term “amino group” refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” can be a primary, secondary, tertiary, or quaternary amino group. An “alkylamino” group includes a monoalkylamino, dialkylamino, and trialkylamino group.

The compounds may contain one or more chiral centers or may otherwise be capable of existing as multiple stereoisomers. It is to be understood that the invention described herein is not limited to any particular stereochemical requirement and that the compounds may be optically pure or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers while including mixtures of stereochemical configuration at one or more other chiral centers.

Similarly, the compounds described herein may include geometric centers, such as cis, trans, E, and Z double bonds. It is to be understood that the invention described herein is not limited to any particular geometric isomer requirement, and that the compounds may be pure, or may be any of a variety of geometric isomer mixtures. It is also to be understood that such mixtures of geometric isomers may include a single configuration at one or more double bonds, while including mixtures of geometry at one or more other double bonds.

### EXPERIMENTAL

The following examples serve to illustrate the present disclosure. The examples are not intended to limit the scope of the claimed invention in any way.

### Abbreviations Used are

DoE: design of experiments; HTE: high throughput experimentation; THF: tetrahydrofuran; 2-MeTHF: 2-methyl tetrahydrofuran; DESI-MS: Desorption electrospray ionization-mass spectrometry; PTFE: polytetrafluoroethylene; MS: molecular sieve; MW: microwave; TMAO; trimethyl N-oxide; EtOAc: ethyl acetate;

### Catalyst Scouting Using Design of Experiments and High Throughput Experimentation (DoE-HTE)

The promising catalysts for the amidation reaction were identified by performing of design of experiments (DoE) using high throughput experimentation (HTE).

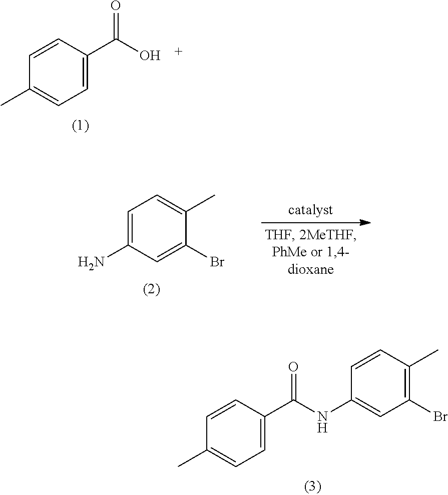

DoE-HTE experiments were conducted to find the best conditions for boronic acid catalysis, for example, four cyclic boranes/boronic acids: triphenyl borate, 2,4-bis(trifluoromethyl) phenyl boronic acid, phenylboronic acid pinacol ester, bis(catecholato)diboron; five aliphatic boronic acids: tetrahydroxy diboron, butylboronic acid, boric anhydride, trimethyl borate, boric acid; and one zirconium catalyst, for example, bis(cyclopentadienyl)zirconium(IV) dichloride).

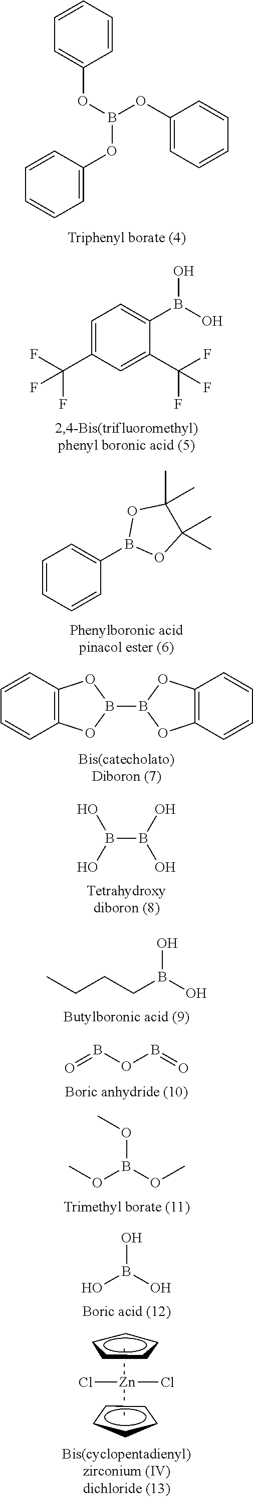

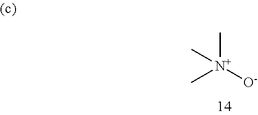

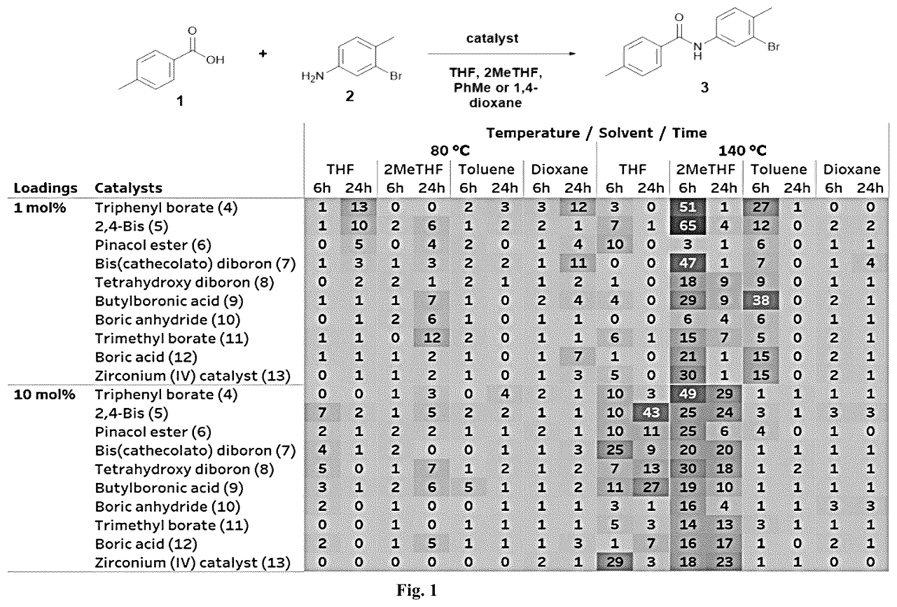

The different solvents, such as THF, 2-MeTHF, toluene, and 1,4-dioxane, were studied for amide coupling efficiency. For the DoE, a 2³ design was used, with loading variation of three factors, temperature, time, and catalyst at two levels: for temperature 80° C. and 140° C.; for time 6 hours and 24 hours; and for catalyst loading 1% and 10%. A total of 320 experiments with solvent controls were performed (FIG. 1) using DESI-MS HTE setup previously reported in Anal. Methods-Uk, 2020, 12, 3654-3669, Org Process Res Dev, 2020, 24, 2240-2251 and ACS Bio Med Chem Au, 2022, 2, 297-306 which are incorporated herein by reference for their teaching regarding the same. In this experimental setup, the reagents were transferred to 96 well reaction blocks using a liquid handling robot, heated the 96 well blocks at specified temperatures and allowed reagents to react for the allotted amount of time, transferred the solutions from processed 96 well blocks to a 384 well daughter plate, spotted the reaction solutions from the 384 well daughter plate to an inert PTFE coated plate and analyzed reactions using DESI-MS. The plate was then sprayed with charged solvent droplets, generating charged microdroplets in which the ions were guided toward the mass spectrometer. The data was analyzed by normalizing the ion counts generated by the noise, in a signal-to-noise ratio (S/N).

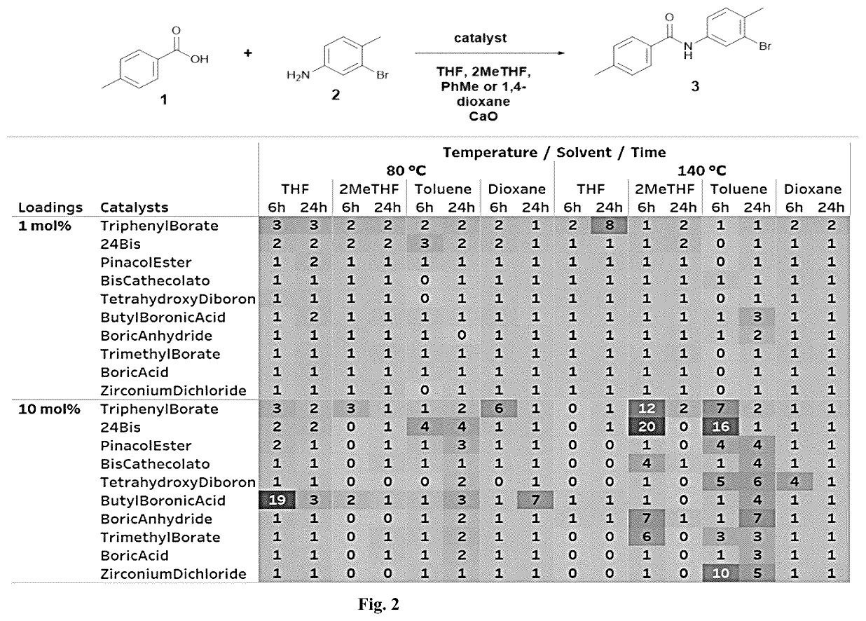

Each DoE-HTE run produced a set of 1280 individual experiments, with four replicates per reaction performed for each of the 320 reaction conditions. The m/z intensities of the replicates were averaged after normalization by the blank and solvent ion intensities (S/N). In the first experiment, without the addition of any drying agents, it was observed that both THF and 1,4-dioxane had lower S/N compared to the product S/N of the reactions performed in 2-MeTHF and toluene (FIG. 2). Since both THF and 1,4-dioxane are infinitely miscible in water and 2-MeTHF and toluene have low water saturation limits, it was reasoned that the accumulation of water in the reaction medium was suppressing product formation in the THF and 1,4-dioxane cases since the rate-limiting step of boronic acid catalytic amidation is the reversible formation of the carboxylic acid-boronic acid ester and elimination of H₂O. Most reactions produced better results at 6 hours compared to 24 hours. As more water accumulates above the separation limit from the solvents, it will interfere with the catalytic cycle, converting the first active intermediate into starting materials.

A second HTE was performed to probe whether the addition of a drying agent could further optimize the reaction. CaO was chosen because the solid Ca(OH)₂ byproduct could be easily filtered from the reaction mixture and not compromise the mass spectrometry analysis. This experiment also gave us 1280 experiments that were again evaluated by DESI-MS. The results revealed a trend in the reaction conditions that can lead to greater yields (FIG. 1 and FIG. 2). It was found that 2,4-bis(trifluoromethyl) phenyl boronic (5) acid produced the highest ion counts, followed by triphenyl borate; similar outcomes were observed for bis(catecholato)diboron, butyl boronic acid, and bis(cyclopentadienyl)zirconium(IV) dichloride. The best solvent was 2-MeTHF, followed by THF and toluene; 1,4-dioxane produced the lowest ion counts. The highest ion counts were observed at 140° C. for 6 hours, containing 10% catalyst loadings.

Solvent selection of a green synthesis route can dramatically impact the safety, cost, environmental impact, and efficiency of the overall process. The water phase separation from 2-MeTHF renders it a more efficient solvent by suppressing water byproduct interference with the catalytic cycle. It is a biorenewable solvent and produced the greatest ion counts in the HTE campaigns.

### Microwave Heating

When the above-optimized conditions were utilized with a Dean-Stark trap or molecular sieves (MSs) for water removal, the reactions were not sufficiently driven to amide formation (Table 5). The addition of N-oxides has been reported in amide bond formation. The use of N-oxides can generate a more active intermediate in a second activation step that increases the rate of conversion to products in the catalytic cycle. The N-oxide used was TMAO (14).

Microwave synthesizers allow for a much faster and homogeneous heating of the solvent mixture compared to conventional heating methods because of the “inside out” phenomenon that has been associated with reduced reaction times (The Royal Society of Chemistry, 2016, 1-33; Catalysts 2020, 10, 991 and Aust. J. Chem. 2009, 62, 16-26). Microwave heating is also more efficient and safer when dealing with temperatures above the boiling point of a solvent, since the vapors are contained within a closed system and the applied power is regulated by a pressure sensor. The next efforts focused on optimizing the conditions for formation of an amide (3) in a microwave synthesizer.

Table 1 shows the reaction yields for microwave optimization experiments and the best results per optimization conditions.

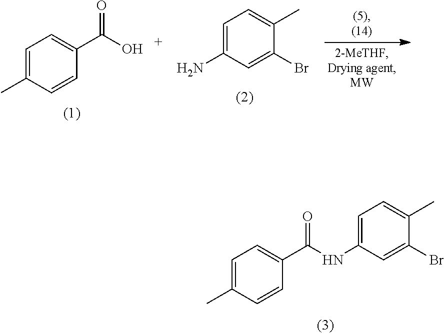

| Catalyst | Additive | | | | |
|---|---|---|---|---|---|
| Loading | Loading | Concentration | Drying | Time | % |
| (mol %) | (mol %) | (mol/L) | agent | (minutes) | Yield |
| 25 | 25 | 0.10 | 4 Å MS | 30 | 25 |
| 25 | 25 | 0.10 | 3 Å MS | 15 | 32 |
| 25 | 25 | 0.25 | 3 Å MS | 10 | 50 |
| 15 | 15 | 0.25 | 3 Å MS | 10 | 34 |
| 50 | 50 | 0.25 | 3 Å MS | 10 | 74 |
| 25 | 25 | 0.25 | 3 Å MS | 30 | 75 |
| 25 | 25 | 0.25 | 3 Å MS | 120 | 86 |

A mixture of 25 mol % (5), 25 mol % (14), 1.1 eq. of acid (1), 1 eq. of amine (2), 0.10 mol/L with 1 g/mL 4 Å MS tested at 180° C. for 30 minutes to evaluate whether 2-MeTHF was a better solvent in a microwave synthesis. A 25% product yield was observed in 30 minutes under these conditions (Table 1 and Table 6). Next evaluated was the role of the drying agent in the reaction by testing with no added drying agent, CaO, 3 Å and 4 Å molecular sieves. The best results were obtained with 3 Å molecular sieves, conferring a 32% yield after 15 min of microwave processing (Table 1 and Table 7). Concentration was found to be pivotal for product formation, with a two-fold increase in product observed as the concentration was increased from 0.10 (24% yield) to 0.25 mol/L (50% yield) for a 10 min reaction period, but no improvement as the concentration increased to 10-fold (Table 1 and Table 8). This may be due to a lack of solubility of the reagents at such a high concentration.

Lower temperatures (100° C. and 140° C.) were less efficient (Table 9), as well as lower acid equivalences (Table 10). Other commercially available n-oxides were utilized (Table 11), as well as other catalysts screened in DoE-HTE, but TMAO (14) and catalyst (5) (Table 12) remained as the best additive and catalyst, respectively. It was hypothesized TMAO acts as a better additive because, for the other N-oxides utilized, there can be charge delocalization, reducing the inductive effect promoted by the additive to improve the electrophilicity of the carbonyl. As for the catalyst, it is known that boronic acids containing electron-withdrawing groups can generate the first activated intermediate faster than the ones without such groups. As for the quantity of drying agent, it was measured in terms of grams of drying agent per milliliter of solvent because of the cylindrical shape of the microwave reaction vessel. The greater the addition of molecular sieves, the more dispersed the solution was in the reaction vessel, and so 1 g/mL proved to be the best ratio for product formation (Table 13).

The effect of the catalyst or additive, either alone or in combination, was evaluated (Table 1 and Table 14). The yields were found to be greater when the catalyst and additive were added in a 1:1 ratio, with higher loadings producing significantly higher yields in just 10 minutes of reaction, with 15 mol % generating 34% yield compared to 74% yield at 50 mol %.

With optimized conditions, the reaction time was increased to 1 hours and 2 hours to allow for more conversion and compared the effect of 15 and 25 mol % loadings at 0.25 M and 0.50 M starting material concentrations. As shown in Table 2 and Table 15, 1 hour increased the product formation to 75% at 0.25M and 25 mol % loading. When the starting material concentrations were increased to 0.50 M, modest yield improvements were observed. The time increase from 1 hour to 2 hours at any concentration produced a marginal improvement of approximately 3%. In order to maintain the reaction as green as possible, studied the effect of reducing the catalyst loadings to 15 mol %. At 0.50 M, 15 mol % catalyst produced a reasonable yield of 63% in 1 h.

The impact of time on the reaction was studied (Table 1 and Table 16). The most significant changes occur within 30 min, with 75% yield, and then small incremental changes occur with time, with a 13% yield improvement observed with an additional 120 minutes of microwave heating.

N-phenylbenzamide (15) was synthesized from aniline and benzoic acid. The reported yield of (6) in Tetrahedron Letters, 2010, 51, 4186-4188 and J Org Chem, 2023, 88, 2832-2840 using a boron catalyst is 0 and 33% respectively. Table 2 shows that the microwave method produces better yields at 25 mol % catalyst and additive loading and increasing reaction times for compound (3), but similar yields for compound (15) with acceptable yields occurring at 15 mol % and 1 hour of reaction.

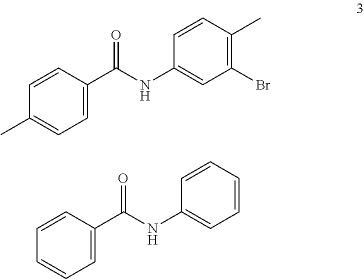

Table 2 shows the reaction yields with 15-25 mol % catalyst (5) and additive (14) loading for (3) and (15) as a function of time.

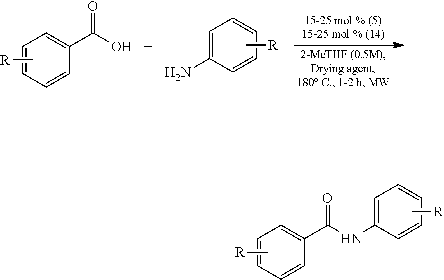

| Catalyst/Additive | Time | (3) % | (6) % |
|---|---|---|---|
| Loadings | (hours) | Yield | Yield |
| 15 mol %/15 mol % | 1 | 63 | 80 |
| 15 mol %/15 mol % | 2 | 66 | 78 |
| 25 mol %/25 mol % | 1 | 82 | 90 |
| 25 mol %/25 mol % | 2 | 86 | 87 |

For the following substrates, the lower catalyst and additive loadings and a reduced reaction time were utilized to give a sense of what is possible with minimal conditions. It was found that some substrates generated excellent yields under these conditions, while others required increased reaction times and/or catalyst loadings. Following are the benzamides formed by 2,4-bis(trifluoromethyl) phenyl boronic acid catalyzed condensation under microwave conditions in the presence of 3 Å molecular sieves and trimethyl N-oxide in 2-MeTHF.

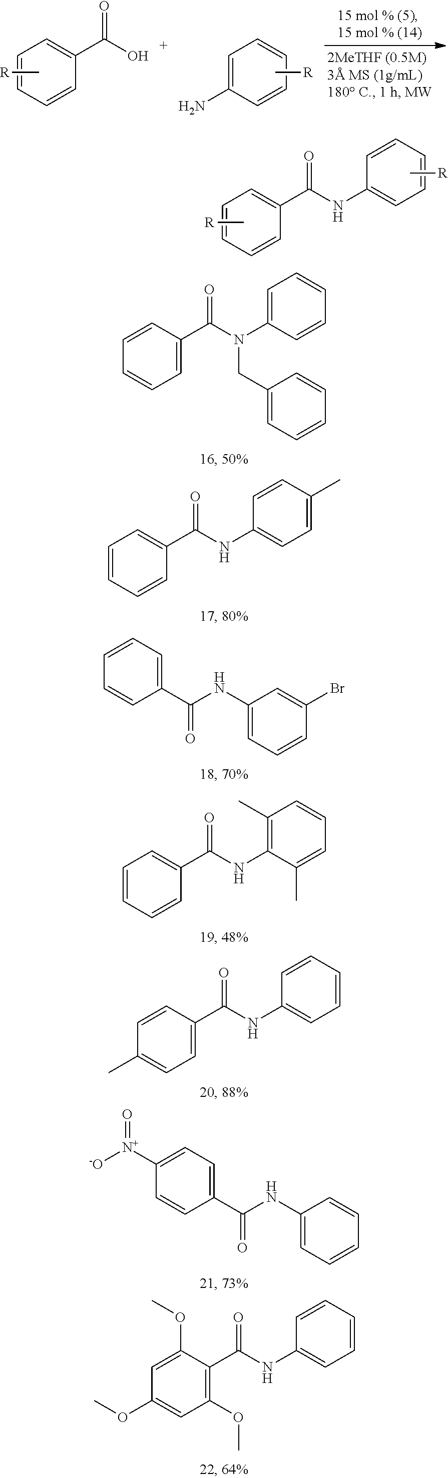

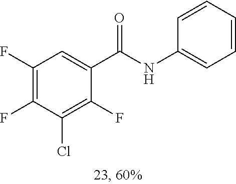 [SMILES: O=C(Nc1ccccc1)c1cc(F)c(F)c(Cl)c1F]

In general, substituted anilines and benzoic acids gave good results, but yields dropped off as either the nucleophile (16 and 19) or electrophile (22 and 23) became more sterically hindered.

Also tested are four examples of unsuccessful couplings between amines and carboxylic acids, such as compounds 24, 25, 26, and 27 where reported yields are 0%, 0%, 0%, and 38%, respectively.

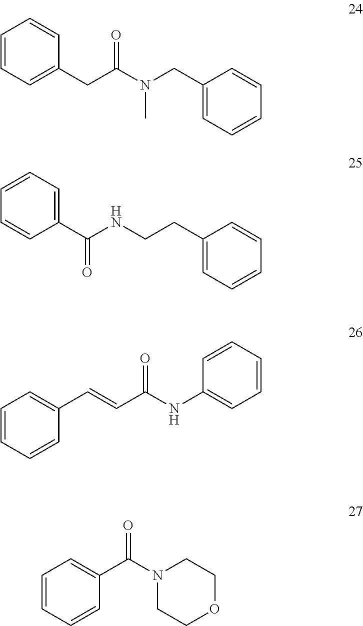 [SMILES: CN(Cc1ccccc1)C(=O)Cc1ccccc1 ; O=C(NCCc1ccccc1)c1ccccc1 ; O=C(/C=C/c1ccccc1)Nc1ccccc1 ; O=C(c1ccccc1)N1CCOCC1]

It was found that the present method was able to produce good to excellent yields of 70%, 95%, 84%, and 86% for 24, 25, 26, and 27, respectively, thus demonstrating the utility of combining catalysis and microwave heating for unreactive aromatic and secondary amine substrates. The time for completion of the reaction was 1 hour.

This method can be utilized to improve atom economy.

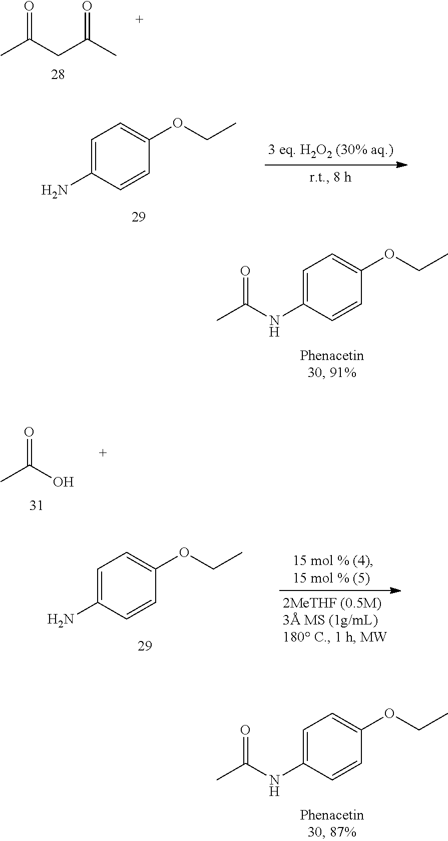

Phenacetin synthesis from the reaction of pentane-2,4-dione (28) and p-phenetidine (29) was reported in Green Chemistry, 2013, 15, 3289 (top) with an atom economy of 70%. The direct amidation of p-phenetidine with acetic acid (31) produced Phenacetin with an 87% yield under catalyzed microwave conditions with an atom economy of 88% (bottom).

### Design of Experiments and High Throughput Experimentation (DoE-HTE)

TABLE 3

| | Solubility of water in | Boiling point |
|---|---|---|
| Solvents | the solvent %)^{1, 2} | (° C.) |
| THF | miscible in all proportions | 65.7 |
| 2-MeTHF | 4 | 80.2 |
| Toluene | 0.033 | 110.6 |
| 1,4-Dioxane | miscible in all proportions | 101 |

### Design of Experiments 2³

TABLE 4

| | Temperature | | Catalyst Loading |
|---|---|---|---|
| Levels | (° C.) | Time (h) | (mol %) |
| −1 | 80 | 6 | 1 |
| 1 | 140 | 24 | 10 |

The DoE-HTE experiments were performed by studying three variables: temperature, time, and catalyst loading, varying each at two levels. The temperature varied at 80 and 140° C.; time for 6 and 24 hours; and ctalyst 1 and 10 mol %, respectively. A total of eight conditions were performed for each of the ten catalysts for the four solvents, resulting in a total of 320 experiments plus solvent controls.

### Batch Reactions

2-MeTHF was selected as a solvent because it produced similar results to THF and toluene and is biorenewable. For 10 mol % loading of 2,4-bis(trifluoromethyl)phenylboronic acid (5), with p-toluic acid (1) and 3-bromo-4-methylaniline (2). The yield obtained was 12% after 24 hours. The catalyst loading was increased to 25 mol % to increase the yield, and the yield obtained was 15%. The use of an n-oxide additive such as TMAO (14, 25 mol %) with 2,4-bis(trifluoromethyl)phenylboronic acid gave 68% product in 24 hours. The solvent was completely evaporated when molecular sieves were added or with a Dean-Stark at 140° C. Table 5 shows the results of batch reactions.

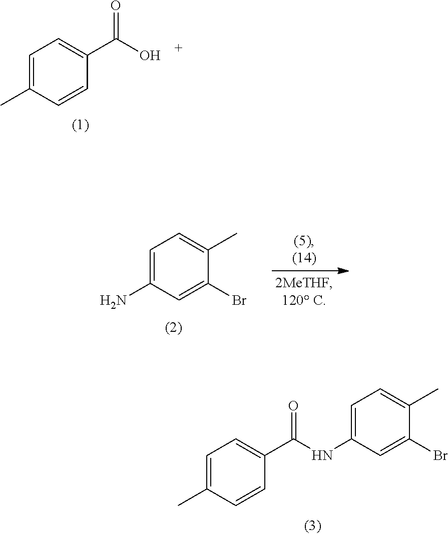

TABLE 5
Batch synthesis

| Catalyst loading | Additive | Time | Yield |
|---|---|---|---|
| (mol %) | loading | (hours) | (%) |
| 10 | 0 | 1 | 0 |
| 10 | 0 | 4 | 0 |
| 10 | 0 | 24 | 12 |
| 25 | 0 | 1 | 2 |
| 25 | 0 | 4 | 5 |
| 25 | 0 | 24 | 15 |
| 25 | 25 | 1 | 9 |
| 25 | 25 | 4 | 18 |
| 25 | 25 | 24 | 68 |

Reaction conditions: compounds (14) and (5): 1.1 eq.; compound (1): 1 eq.; compound (2): 0.10 mol/L; 1 g/mL 4 Å MS and 120° C.

### Microwave Method Development

It was hypothesized that one of the reasons for the success of the reaction in DoE-HTE compared to batch reactions is that the reactions were done in sealed tubes, and 2-MeTHF could not escape, since reactions were performed well above its boiling point of 80.2° C. Microwave reactors are able to efficiently heat solvents above their boiling point. The effect of various factors on the yield of the reaction was studied. The factors evaluated were different solvents (Table 6), drying agents (Table 7), concentrations (Table 8), temperatures (Table 9), molar ratios of acid and amine (Table 10), additives (Table 11), catalysts (Table 12), amounts of drying agent (Table 13), catalyst and additive loadings (Table 14), concentration of time, loading and time (Table 15) and time (Table 16)

TABLE 6
Solvents

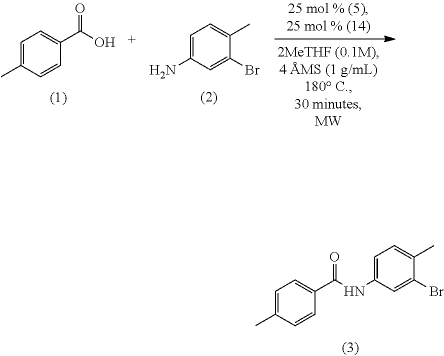

| Solvent | Yield (%) |
|---|---|
| DMAc | 17 |
| DMF | 11 |
| ACN | 11 |
| 2-MeTHF | 25 |

Reaction conditions: compounds (14) and (5): 25 mol %; compound (1): 1.1 eq; compound (2): 1 eq.; 2MeTHF: 0.10 mol/L; 1 g/mL 4Å MS, and 180° C. for 30 minutes.

TABLE 7
Drying Agents (DA)

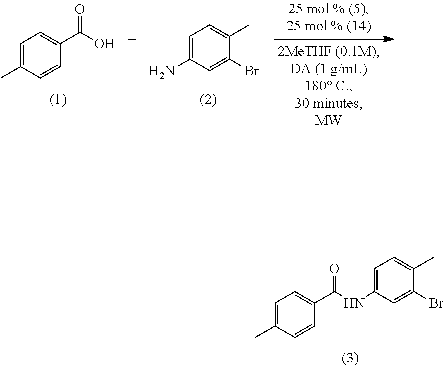

| Drying Agent | Yield (%) |
|---|---|
| None | 0 |
| 3Å MS | 32 |
| 4Å MS | 20 |
| CaO | 5 |

Reaction conditions: compounds (14) and (5): 25 mol %; 1.1 eq compound (1); 1 eq. compound (2); 0.10 mol/L; 2MeTHF: 0.1M; DA: 1 g/mL and 180° C. for 15 minutes.

TABLE 8
Concentration of Substrates

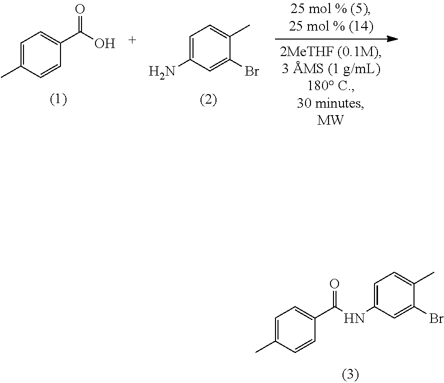

| Concentration (M) | Yield (%) |
|---|---|
| 0.10 | 24 |
| 0.25 | 50 |
| 1.0 | 18 |

Reaction conditions: compounds (14) and (5): 25 mol %; 1.1 eq compound (1); 1 eq. compound (2); 0.10 mol/L; 2MeTHF: 0.1-1.0M; 3Å MS: 1 g/mL and 180° C. for 10 minutes.

TABLE 9
Temperature

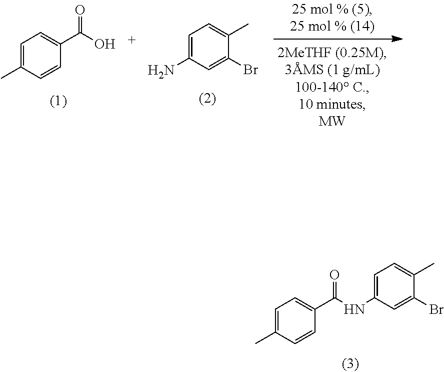

| Temperature (° C.) | Yield (%) |
|---|---|
| 100 | 12 |
| 140 | 0 |

Reaction conditions: compounds (14) and (5): 25 mol %; 1.1 eq. compound (1); 1 eq. compound (2); 0.25 mol/L, 2MeTHF; 3Å MS: 1 g/mL and 100-140° C. for 10 minutes.

TABLE 10
Equivalence

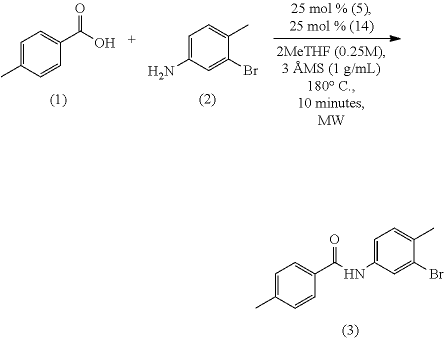

| Equivalence acid: amine | Yield (%) |
|---|---|
| 1:1 | 27 |
| 1:1.1 | 19 |

Reaction conditions: compounds (14) and (5): 25 mol %; 1.1 eq. compound (1); 1 eq. compound (2); 0.25 mol/L, 2MeTHF; 3Å MS: 1 g/mL and 180° C. for 10 minutes.

TABLE 11
Additive

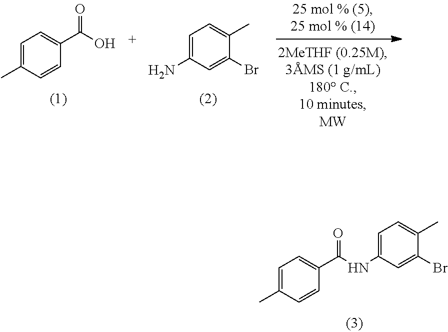

| Additive | Yield (%) |
|---|---|
| Trimethylamine N-oxide (TMAO) | 50 |
| 4-(Dimethylamino)pyridine n-oxide hydrate (DMAPO) | 6 |
| N-Methylmorpholine (NMO) | 12 |
| N-tert-Butyl-alpha-(4-pyridyl-1-oxide)nitrone | 3 |
| Resazurin sodium salt | 1 |

Reaction conditions: compound (14) and additive: 25 mol %; 1.1 eq. compound (1); 1 eq. compound (2); 0.25 mol/L, 2MeTHF; 3Å MS: 1 g/mL and 180° C. for 10 minutes.

TABLE 12
Catalyst

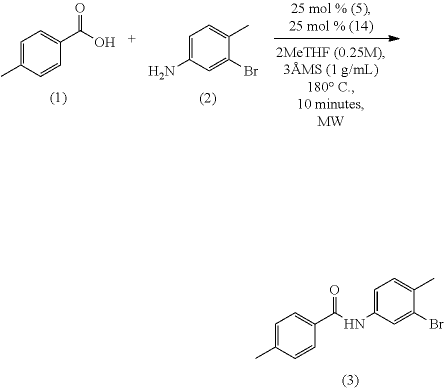

| Catalyst | Yield (%) |
|---|---|
| Triphenyl boronic acid | 17 |
| Butyl boronic acid | 0 |

Reaction conditions: compound (14) and catalyst: 25 mol %; 1.1 eq. compound (1); 1 eq. compound (2): 0.25 mol/L, 2MeTHF; 3 Å MS: 1 g/mL and 180° C. for 10 minutes.

TABLE 13
Drying Agent Quantity

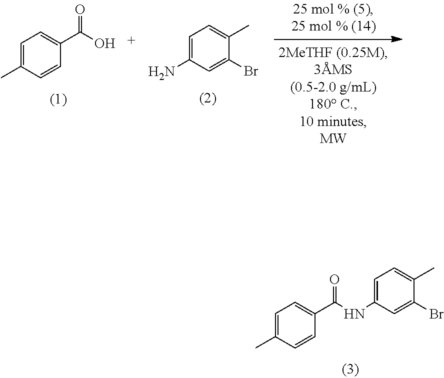

| Drying Agent (g/ mL solvent) | Yield (%) |
|---|---|
| 0.5 | 32 |
| 1.5 | 36 |
| 2 | 17 |

Reaction conditions: compounds (14) and (5): 25 mol %; 1.1 eq; compound (1); 1 eq.; compound (2); 0.25 mol/L, 2MeTHF; 3Å MS: 0.5-2.0 g/mL and 180° C. for 10 minutes.

TABLE 14
Catalyst and Additive Loading

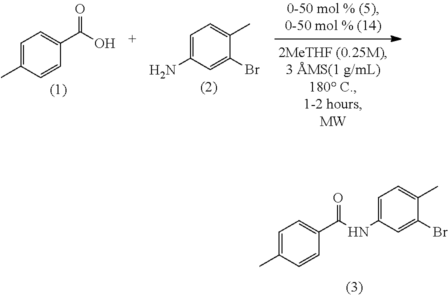

| Catalyst loading (mol%) | Additive loading (mol%) | Yield (%) |
|---|---|---|
| 25 | 0 | 9 |
| 25 | 5 | 10 |
| 25 | 15 | 17 |
| 25 | 25 | 55 |
| 0 | 25 | 3 |
| 5 | 5 | 6 |
| 15 | 25 | 30 |
| 5 | 25 | 14 |
| 15 | 15 | 34 |
| 5 | 15 | 12 |
| 5 | 5 | 14 |
| 50 | 50 | 74 |

Reaction conditions: compounds (14) and (5): 0-50 mol %; 1.1 eq compound (1); 1 eq. compound (2); 0.25 mol/L, 2MeTHF; 3Å MS: 1.0 g/mL and 180° C. for 10 minutes.

TABLE 15
Concentration, loading, and time optimization

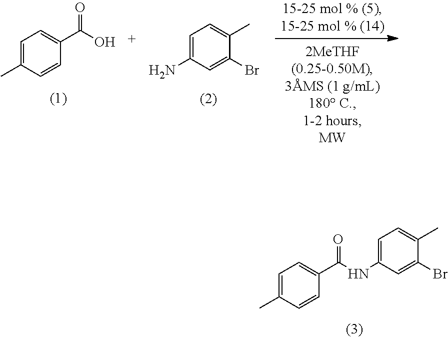

| | | Time | Yield |
|---|---|---|---|
| Concentration (M) | Loading mol% | (h) | (%) |
| 0.25 | 15 | 1 | 45 |
| 0.25 | 15 | 2 | 54 |
| 0.50 | 15 | 1 | 63 |
| 0.50 | 15 | 2 | 66 |
| 0.25 | 25 | 1 | 75 |
| 0.25 | 25 | 2 | 76 |
| 0.50 | 25 | 1 | 82 |
| 0.50 | 25 | 2 | 86 |

Reaction conditions: compounds (14) and (5): 15-25 mol %; 1.1 eq compound (1); 1 eq compound (2); 2MeTHF: 0.25-0.50M; 3Å MS: 1.0 g/mL and 180° C. for 1-2 hours.

TABLE 16
Time

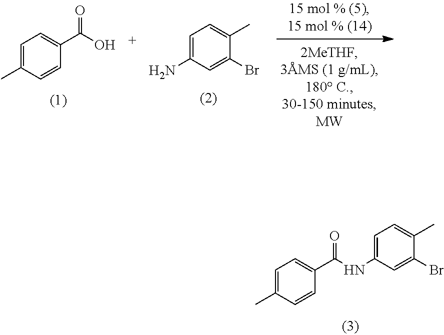

| Time (min) | Yield (%) |
|---|---|
| 30 | 75 |
| 60 | 82 |
| 120 | 86 |
| 150 | 88 |

Reaction conditions: compounds (14) and (5): 15-25 mol %, 1.1 eq; compound (1): 1 eq.; compound (2): 0.50 mol/L; 2MeTHF: 0.50M; 3 Å MS: 1.0 g/mL and 180° C. for 30-150 minutes.

The best conditions generalized as 25 mol % (14), 25 mol % (5), 1.1 eq. (1), 1 eq. (2), 0.50 mol/L, 2-MeTHF, 1 g/mL 3 Å MS, 180° C. 1 h. For maximum yield, reactions could be performed for 120-150 min, and for lower use of catalyst, reactions can be performed at 15 mol % catalyst and additive loadings.

### General Procedure

Amine (0.50 mol/L, 1.07 mmol), acid (1.18 mmol), catalyst (0.16 mmol or 0.27 mmol), and additive (15 mol % or 25 mol %) and 2-MeTHF (2 mL) were added to a 10 mL CEM microwave vial previously charged with a stir bar and swirled to dissolve all contents. Then 2 g (1 g/mL) of 3 Å molecular sieves (previously activated overnight, heated to 120° C. in a round bottom flask with an oil bath under high vacuum) were added to the vial, and the vial was capped. The CEM Discover® 1.0 synthesizer was programmed with a simple program with a fixed temperature for 180° C., and the reaction was performed for the allotted time for all experiments.

### Substrate Scope Reactions:

Reactions were performed at 0.50 mol/L, 0.54 mmol of amine, 0.59 mmol of acid, 0.08 mmol of catalyst and additive (15 mol %), and 1 mL of 2-MeTHF were added to a 10 mL CEM microwave vial previously charged with a stir bar and swirled to dissolve all contents. Then 1 g (1 g/mL) of 3 Å molecular sieves (previously activated) was added to the vial, and the vial was capped. The CEM Discover® 1.0 synthesizer was programmed with a simple program with a fixed temperature for 180° C. and the reaction was performed for 1 hour.

After reaction completion, the contents were filtered to remove the molecular sieves, and the vial was washed three times with EtOAc and then filtered. Purification occurred via extraction or column chromatography. For extraction, an EtOAc/2-MeTHF mixture was washed with 1M NaOH, followed by 1M HCl solution; the organic layer was dried with Na₂SO₄, filtered, and the organic phase evaporated to dryness using a rotary evaporator. For column chromatography, the EtOAc/2-MeTHF mixture was evaporated to dryness using a rotary evaporator and loaded onto a column that was eluted with an 80/10 hexanes/EtOAc to 0/100 hexanes/EtOAc gradient. Pure fractions were evaporated to dryness using a rotary evaporator.

### EXAMPLES

### N-(3-Bromo-4-methylphenyl)-4-methylbenzamide (3)

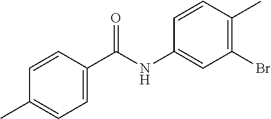 [SMILES: Cc1ccc(C(=O)Nc2ccc(C)c(Br)c2)cc1]

¹H NMR (500 MHz, CDCl3): δ [ppm]=7.87 (d, 1H), 7.85 (s, 1H), 7.74 (d, 2H), 7.47 (dd, 1H), 7.25 (d, 2H), 7.18 (d, 1H), 2.41 (s, 3H), 2.36 (s, 3H).

¹³C NMR (500 MHz, CDCl3): δ [ppm]=161.63, 142.56, 136.84, 133.84, 131.73, 130.82, 129.47, 127.04, 124.80, 123.94, 119.21, 22.30, 21.52.

HRMS [M+H]+ calculated for 304.03. found: 304.0356.

### N-Phenylbenzamide (15)

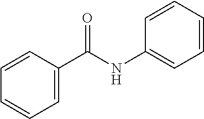 [SMILES: O=C(Nc1ccccc1)c1ccccc1]

¹H NMR (500 MHz, CDCl3): δ [ppm]=7.87 (d, 2H), 7.81 (s, 1H), 7.64 (d, 2H), 7.55 (d, 1H), 7.49 (t, 2H), 7.38 (t, 2H), 7.16 (t, 1H).

This compound is reported in J. Org. Chem. 2023, 88, 2832-2840.

### N-Benzyl-N-phenylbenzamide (16)

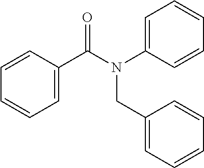 [SMILES: O=C(c1ccccc1)N(Cc1ccccc1)c1ccccc1]

¹H NMR (500 MHz, CDCl3): δ [ppm]=7.34-7.28 (m, 6H), 7.25-7.21 (m, 2H), 7.17-7.07 (m, 5H), 6.90 (d, 2H), 5.14 (s, 1H).

This compound is reported in Asian J. Org. Chem. 2020, 9, 364-367.

### N-(4-Methylphenyl)benzamide (17)

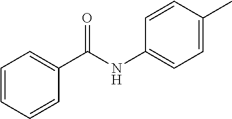 [SMILES: Cc1ccc(NC(=O)c2ccccc2)cc1]

¹H NMR (400 MHz, CDCl3): δ [ppm]=7.86 (d, 2H), 7.73 (s, 1H), 7.57-7.46 (m, 5H), 7.18 (d, 2H), 2.34 (s, 3H).

This compound is reported in Asian J. Org. Chem. 2018, 7, 683-687.

### N-(3-Bromophenyl)benzamide (18)

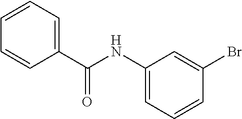 [SMILES: O=C(Nc1cccc(Br)c1)c1ccccc1]

¹H NMR (400 MHz, CDCl3): δ [ppm]=7.93 (s, 1H), 7.89-7.86 (m, 2H), 7.79 (s, 1H), 7.61-7.59 (m, 2H), 7.54-7.49 (m, 2H), 7.31-7.29 (m, 1H), 7.26-7.22 (m, 1H).

This compound is reported in Tetrahedron 2016, 436-441.

### N-(2,6-Dimethylphenyl)benzamide (19)

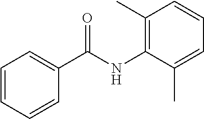 [SMILES: Cc1cccc(C)c1NC(=O)c1ccccc1]

¹H NMR (500 MHz, CDCl3): δ [ppm]=8 [ppm]=7.95-7.92 (m, 2H), 7.59-7.56 (m, 1H), 7.53-7.50 (m, 2H), 7.36 (s, 1H), 7.17-7.12 (m, 3H), 2.30 (s, 6H).

This compound is reported in Org. Lett. 2017, 19, 2158-2161.

### 4-Methyl-N-phenylbenzamide (20)

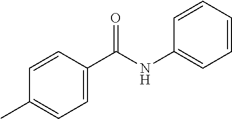 [SMILES: Cc1ccc(C(=O)Nc2ccccc2)cc1]

¹H NMR (500 MHz, CDCl3): δ [ppm]=7.77 (d, 3H), 7.64 (d, 2H), 7.37 (t, 2H), 7.29 (d, 2H), 7.15 (t, 1H), 2.43 (s, 3H).

This compound is reported in Green Chem. 2014, 16, 2443-2447.

### 4-Nitro-N-phenylbenzamide (21)

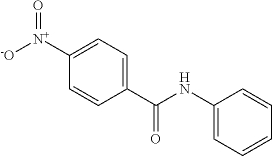

¹H NMR (500 MHz, d⁶-DMSO): δ [ppm]=10.53 (s, 1H), 8.36 (d, 2H), 8.17 (d, 2H), 7.76 (d, 2H), 7.36 (t, 2H), 7.13 (t, 1H).

This compound is reported in Green Chem. 2014, 16, 2443-2447.

### 2,4,6-Trimethoxy-N-phenylbenzamide (22)

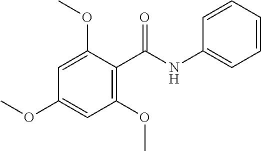 [SMILES: COc1cc(OC)c(C(=O)Nc2ccccc2)c(OC)c1]

¹H NMR (400 MHz, CDCl3): δ [ppm]=7.64 (d, 2H), 7.49 (s, 1H), 7.33 (t, 2H), 7.10 (t, 1H), 6.14 (s, 2H), 3.88-3.80 (m, 9H).

This compound is reported in Chem. Biol. Drug Des. 2016, 88, 820-831.

### 3-Chloro-2,4,5-trifluoro-N-phenylbenzamide (23)

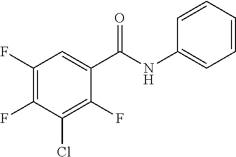 [SMILES: O=C(Nc1ccccc1)c1cc(F)c(F)c(Cl)c1F]

¹H NMR (500 MHz, d⁶-DMSO): δ [ppm]=10.56 (s, 1H), 7.93-7.88 (m, 1H), 7.67 (d, 2H), 7.36 (t, 2H), 7.13 (t, 1H).

¹³C NMR (500 MHz, d⁶-DMSO): δ [ppm]=160.31, 138.87, 129.38, 124.81, 120.30, 116.82, 116.66.

HRMS [M+H]+ calculated for 286.02. found: 286.0253.

### N-Benzyl-N-methyl-2-phenylacetamide (24)

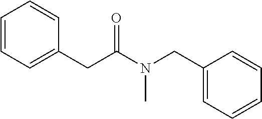 [SMILES: CN(Cc1ccccc1)C(=O)Cc1ccccc1]

¹H NMR (500 MHz, CDCl3): δ [ppm]=As described in the reference, the presence of two rotamers with a ratio of 1:1.3 was observed. 7.36-7.21 (m, 17H), 7.10 (d, 2H), 4.61 (s, 2H), 4.53 (s, 2H), 3.79 (s, 2H), 3.76 (s, 2H), 2.96 (s, 3H), 2.90 (s, 3H).

This compound is reported in Tetrahedron Letters. 2015, 56, 4527-4531.

### N-Phenethylbenzamide (25)

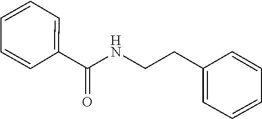 [SMILES: O=C(NCCc1ccccc1)c1ccccc1]

¹H NMR (500 MHz, CDCl3): δ [ppm]=7.69 (d, 2H), 7.50-7.47 (m, 1H), 7.42-7.39 (m, 2H), 7.35-7.32 (m, 2H), 7.26-7.24 (m, 3H), 6.18 (s, 1H), 3.72 (q, 2H), 2.94 (t, 2H).

This compound is reported in Tetrahedron Letters. 2015, 56, 199-202.

### N-Phenylcinnamamide (26)

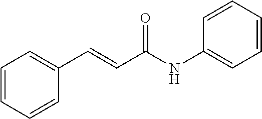 [SMILES: O=C(/C=C/c1ccccc1)Nc1ccccc1]

¹H NMR (400 MHz, CDCl3): δ [ppm]=7.76 (d, 1H), 7.64-7.61 (m, 2H), 7.54-7.52 (m, 2H), 7.42-7.33 (m, 6H), 7.15-7.12 (m, 1H), 6.58-6.54 (d, 1H).

This compound is reported in Chem. Cent. J. 2017, 11, 87.

### Morpholino(phenyl)methanone (27)

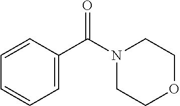 [SMILES: O=C(c1ccccc1)N1CCOCC1]

¹H NMR (500 MHz, CDCl₃): δ [ppm]=7.41 (s, 5H), 3.77-3.46 (m, 8H).

This compound is reported in J. Org. Chem. 2023, 88, 2832-2840.

### Phenacetin [N-(4-Ethoxyphenyl)acetamide)] (30)

¹H NMR (500 MHz, CDCl₃): δ [ppm]=7.53-7.47 (m, 1H), 7.36 (d, 2H), 6.82 (d, 2H), 3.99 (q, 2H), 2.12 (s, 3H), 1.38 (t, 3H).

This compound is reported in Green Chem. 2013, 15, 3289.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

What is claimed is:

1. A method of a catalytic amidation, which method comprises reacting an amine with an acid using a catalyst selected from a boronic acid, a borate, a boric acid, a boronic acid ester, and a Lewis acid, optionally in combination with amine N-oxide additive, using a microwave radiation.

2. The method of claim 1, wherein the amine is an aryl amine or an alkyl amine.

3. The method of claim 1, wherein the acid is an aryl acid or an alkyl acid.

4. The method of claim 1, wherein the catalyst is selected from triphenyl borate, 2,4-bis(trifluoromethyl) phenyl boronic acid, phenylboronic acid pinacol ester, bis(catecholato)diboron, tetrahydroxy diboron, butylboronic acid, boric anhydride, trimethyl borate and boric acid.

5. The method of claim 4, wherein the catalyst is 2,4-bis(trifluoromethyl)phenylboronic acid.

6. The method of claim 1, wherein the catalyst is bis(cyclopentadienyl)zirconium(IV) dichloride.

7. The method of claim 1, wherein the additive is selected from trimethylamine N-oxide, isoquinoline N-oxide, pyridine N-oxide, 2-aminopyridine N-oxide, 2-(4-methoxyphenyl)pyridine N-oxide, 2-mercaptopyridine N-oxide, 2-methyl-4-nitropyridine N-oxide, 2-pyridinol 1-oxide, 2,6-dichloropyridine N-oxide, 4-chloropyridine N-oxide, 4-cyanopyridine N-oxide, 4-methylpyridine N-oxide, 4-phenylpyridine N-oxide, 4-(dimethylamino)pyridine N-oxide, 4-methylmorpholine N-oxide, N-tert-butyl-alpha-(4-pyridyl-1-oxide)nitrone, N-tert-butyl-alpha-4-phenylnitrone, and Resazurin sodium salt.

8. The method of claim 7, wherein the additive is trimethylamine N-oxide.

9. The method of claim 1, which further comprises a step of removing or complexing water.

10. The method of claim 9, wherein the water is removed by using a drying agent selected from a molecular sieve and CaO.

11. The method of claim 10, wherein the drying agent is a molecular sieve.

12. The method of claim 1, wherein the method further comprises the use of an organic solvent.

13. The method of claim 12, wherein the organic solvent is 2-methyl tetrahydrofuran.

14. The method of claim 1, wherein the amount of the catalyst is loaded in a range from about 0.5 mol % to about 50 mol %.

15. The method of claim 1, wherein the amount of the catalyst and additive used is in a ratio of about 1:1 (such as 1:1)

16. The method of claim 1, wherein the amine and acid is used in a ratio of about 1:1 (such as 1:1).

17. The method of claim 1, wherein the method is carried out at in a temperature range from about 20° C. to about 180° C.

18. The method of claim 1, wherein the method is carried out in a time period of about 1 min. to about 150 min.
